(19) United States
(12) Patent Application Publication
(10) Pub. No.: US 2014/0272947 A1
(43) Pub. Date: Sep. 18, 2014

(54) METHODS AND COMPOSITIONS FOR SCREENING MODULATORS OF ONCOGENE-INDUCED SENESCENCE

(71) Applicants: Fox Chase Cancer Center, Jenkintown, PA (US); The Wistar Institute of Anatomy and Biology, Philadelphia, PA (US)

(72) Inventors: Rugang Zhang, Elkins Park, PA (US); Benjamin Guy Bitler, Elkins Park, PA (US); Jeffrey Peterson, Cheltenham, PA (US)

(73) Assignees: Fox Chase Cancer Center, Jenkintown, PA (US); The Wistar Institute of Anatomy and Biology, Philadelphia, PA (US)

(21) Appl. No.: 14/202,909

(22) Filed: Mar. 10, 2014

Related U.S. Application Data
(60) Provisional application No. 61/789,328, filed on Mar. 15, 2013.

(51) Int. Cl. C12Q 1/34

(52) U.S. Cl. CPC C12Q 1/34
USPC 435/6.1; 435/18

(57) ABSTRACT

A high-throughput method for identifying a compound or biomolecule that modulates cell senescence involves simultaneously measuring in a cell population exposed to the test compound or biomolecule, the expression of a senescence marker and cell number, wherein each well contains a single test compound; and determining from said simultaneous measurements whether the test compound increases or decreases cell senescence. In various embodiments, the method is useful is identifying compounds that delay the aging process of normal healthy cells, or identifying a compound useful as a tumor suppressor or identifying a compound useful in the treatment of cancer.

### CROSS REFERENCE TO RELATED APPLICATIONS

This application claims the benefit of the priority of U.S. Provisional Patent Application No. 61/789,328, filed Mar. 15, 2013, which is incorporated herein by reference.

### STATEMENT REGARDING FEDERALLY SPONSORED RESEARCH OR DEVELOPMENT

This invention was made with government support under Grant Nos. R01CA160331 and R01GM083025 and CA010815 awarded by the National Institutes of Health and OC093420 awarded by the Department of Defense. The government has certain rights in this invention.

### BACKGROUND OF THE INVENTION

Aberrant oncogene activation is an important driver of cellular transformation. Initial atypical oncogene activation that occurs in primary cells, such as RAS in mammalian cells, typically triggers cellular senescence, a state of stable cell growth arrest¹¹⁻³. Oncogene-induced senescence (OIS) is an important tumor suppressive mechanism or pathway, and suppression of OIS contributes to cell transformation and promotes tumorigenesis⁴. Oncogenes trigger senescence through a multitude of incompletely understood downstream signaling events that frequently involve protein kinases. For example, oncogenic RAS or BRAF triggers senescence of melanocytes, which results in formation of benign nevi and thus suppresses melanoma development⁵⁻⁷. The RAS oncogene is mutated in a number of cancer types⁸. Oncogenic RAS has been extensively studied in the context of OIS, where it triggers senescence by a cascade of kinases³.

Senescent cells exhibit several distinctive morphological characteristics and molecular markers, including a large, flat morphology, decrease in cell proliferation and expression of senescence-associated β-galactosidase activity (SA-β-gal)⁹⁻¹⁰. Expression of SA-β-gal activity is considered a universal marker of senescent cells¹¹. In addition, senescence induced by oncogenic RAS is also characterized by domains of transcriptionally silenced heterochromatin, known as senescence-associated heterochromatin foci (SAHF)¹². SAHF contribute to senescence by silencing proliferation-promoting genes such as E2F target genes¹³. Inactivation of tumor suppressors such as p53 and p16 inhibits OIS³.

Due to the importance of OIS in tumor suppression, compounds/molecules that regulate OIS not only serve as useful tools in studying OIS, but may also lead to the identification of new tumor suppressors.

### SUMMARY OF THE INVENTION

Described herein is a method providing an effective mechanism for the screening and identification of tumor suppressors and other modulators of cell senescence.

In one aspect, a high-throughput method is provided for identifying a compound or biomolecule that modulates cell senescence. This method involves simultaneously measuring in a cell population in multiwall format, the expression of a senescence marker and cell number. In one embodiment of this method, each well contains a single test compound. The method also involves determining from a correlation of the simultaneous measurements whether a test compound or biomolecule increases or decreases cell senescence.

In another aspect, the method involves identifying cells showing a decrease in expression of the senescence marker with an increase in cell number as compared to the negative control and identifying the test compound or biomolecule in those cell populations as promoting cell growth and suppressing cell senescence.

In another aspect the method involves identifying cells showing an increase in expression of the senescence marker and a decrease in cell number as compared to the negative control and identifying the test compound or biomolecule in those cell populations as inhibiting cell growth and promoting senescence.

In one aspect, the method for screening biomolecules permit identification of novel oncogene induced senescence (OIS) regulators. In one embodiment, a method for identifying target proteins required for RAS-induced senescence is provided. In another embodiment, method disclosed herein is useful for elucidating signaling pathways mediating OIS by targeting critical pathway components.

In still another aspect, the methods described herein are computer-implemented.

Other aspects and advantages of these compositions and methods are described further in the following detailed description of the preferred embodiments thereof.

### BRIEF DESCRIPTION OF THE DRAWINGS

FIG. 1 is a flow chart showing the high throughput method or senescence screen setup and analysis. Primary fibroblasts (IMR90) were transduced with a H-RasG12V encoding retrovirus over two days (Day −1 and 0). Cells were then plated into 96-well plates (1,000 cells/well), selected with puromycin (1 μg/mL) for two days and at the end of the second day, each well was treated with a biomolecule from the kinase inhibitor library at a uniform concentration (250 nM). Cells were incubated for seven additional days to allow for senescence and then fixed in a formaldehyde/glutaraldehyde solution. To visualize the SA-β-gal activity and determine cell number, fixed cells were incubated with the β-galactosidase substrate dye, X-gal, and labeled with the fluorescent nuclear stain, DAPI.

FIG. 2A is a representative bright-field image from screen output generated by using the IMAGEXPRESS MICRO high-content imaging system on the wells described in FIG. 1. Each well was systematically imaged 8 times: 4-bright-field images and 4-fluorescence images. Note the “mask” (light pixels) applied to bright-field images indicating the quantified SA-β-gal positive area.

FIG. 2B is a bar graph showing the quantification of SA-β-gal positive area between Control and H-RasG12V cells (*p=0.029) of the wells of FIG. 1

FIG. 2C are representative fluorescence images from screen generated by using the IMAGEXPRESS MICRO high-content imaging system on wells described in FIG. 1

FIG. 2D is a bar graph showing the quantification of nuclei between Control and H-RasG12V cells (**p=0.0034) of the wells of FIG. 1.

FIG. 3 is a scatterplot showing the identification of senescence suppressing kinase inhibitors. Both the nuclei number and SA-β-Gal area were normalized to DMSO-treated control wells (dot in center of solid crossed bars). The normalized numbers of nuclei (a surrogate of cell number) and SA-β-gal area were graphed. The delineation (dotted lines) separates the graph into quadrants, and placement of the lines was determined from the standard deviation of DMSO control wells (the solid crossed bars). The lower right quadrant indicates kinase inhibitors that have significantly more nuclei and less SA-β-gal area (gray or blue dots). Arrow indicates inhibitor used as negative control in subsequent validation experiments.

FIG. 4A are micrographs showing the validation of kinase inhibitors that significantly inhibit SA-β-gal activity. Primary fibroblasts (IMR90) were transduced with a control or H-RasG12V encoding retrovirus. Cells were plated into 6-well plates (15,000 cells/well), selected with puromycin (1 μg/mL) for two days and at the end of the second day treated with 250 nM of kinase inhibitors, CAS nos. 175178-82-2 or 648449-76-7, or with DMSO. Cells were incubated for seven additional days to allow for senescence and then fixed. SA-β-gal activity was visualized and nuclei were labeled with DAPI.

FIG. 4B is a bar graph showing the quantification of SA-β-gal positive cells. (Cells counted >300 per condition). Kinase inhibitor designation is CAS number under the X axis. Average percentages and p-values are provided in Table 2. Experiments were performed in triplicate (Error bars=standard error).

FIG. 5A are micrographs showing that senescence-suppressing compounds also inhibit SAHF, an additional marker of senescence. Primary fibroblasts (IMR90) were transduced with a control or H-RasG12V encoding retrovirus. Cells were plated into 6-well plates (15,000 cells/well), selected with puromycin (1 μg/mL) for two days and at the end of the second day treated with 250 nM of kinase inhibitors, CAS nos. 175178-82-2 or 648449-76-7 or with DMSO. Cells were incubated for seven additional days to allow for senescence and then fixed. Nuclei were labeled with DAPI, and nuclei containing punctate spots (arrows) were counted as SAHF positive.

FIG. 5B is a bar graph showing quantification of SAHF positive cells (Cells counted >300). Order of inhibitors determined by SA-β-gal activity shown in FIG. 3. Inhibitor designation is CAS number. Average percentages and p-values are in Table 2. Experiments were performed in triplicate (Error bars=standard error).

### DETAILED DESCRIPTION OF THE INVENTION

A high-throughput method for identifying a compound or biomolecule that modulates cell senescence is described herein. The method involves simultaneously measuring in a cell population exposed to the compound or biomolecule the expression of a senescence marker and cell number to reflect changes in proliferation in the same population of cells. This method thus monitors two hallmarks of senescence, senescence associated β-galactosidase activity expression and inhibition of proliferation simultaneously, identifying SA-β-gal activity as the primary measure of senescence, while eliminating artifactual hits due to decreased cell number using nuclear staining. The method, in whole or in part, may be automated or performed by a computer.

In one embodiment, the method employs a multi-well format. In one embodiment, each well or a designated series or section of wells on the plate contains a single test compound selected from a number of compounds. In another embodiment, multiple wells of the plate contain the same compound. From the simultaneous measurements, a determination may be made to identify a test compound that increases or decreases cell senescence.

Cells useful in this method include primary mammalian cells transduced with an oncogene or mammalian cancer cells that naturally express an oncogene. The selection of the oncogene is not a limitation of this method. Exemplary oncogenes are RAS, B-raf, PI3K/AKT or loss of PTEN, among other known oncogenes. Other suitable cells for examination in this method are naturally senescent healthy mammalian cells. Such transduced, cancer or healthy mammalian cells may include fibroblasts, melanocytes, hepatocytes or epithelial cells, among others. In an exemplary embodiment of the method, the cells are cancer cells, such as melanoma cells. However, other cancer cells may be readily selected for use in this method, and such cells are not a limitation of the method.

As used herein, the term “mammalian” includes primarily humans, domestic animals, such as dogs and cats, horses, other farm animals, and laboratory animals (e.g., mice, rats) and the like.

The “test compounds” or biomolecules for screening in this method may be any chemical molecule or biomolecule desired to be assessed for its effect on cell senescence. In one embodiment, the compounds are conventional described as small molecules. In the examples below, for proof of principle, the molecules employed are known kinase inhibitors from a library of such molecules. It is understood that any chemical or biological compound may be screened and assayed by this method.

Additionally conventional multi-well formats may be used, including without limitation, 96-well plates or 384-well plates. The amounts and number of cells (e.g., about 1000 cells/well) and reagents applied to the wells are also conventional, and may be selected by one of skill in the art.

In one embodiment of the method, the cells in each well are treated or incubated with a single test compound per well for a time suitable for modulation of senescence in the wells. The amount of test compound added per well is a uniform concentration. In one embodiment, such a uniform concentration ranges from 1 nM to 10 μM of compound per well. In one embodiment, the concentration is at least 2, 10, 50, 100, 250, 500, 750 or 1000 nM. In another embodiment, the concentration is at least 1, 5 or 10 μM. Still other amount intervening between any two of the amounts identified is anticipated to be useful. Generally such an incubation time and temperature may be conventionally selected by one of skill in the art. In one embodiment, the time for modulation may be from 24 hours to about 7 days, depending upon the test compound employed. A desired temperature is one suited for cell culturing, such as 37° C. (also suitable for the X-gal substrate). Optionally, the cells containing the single test compound are fixed and washed prior to further treatment or incubation with additional components of the method. Washing may be accomplished with water, buffer or saline. Normal cell fixatives, such as 4% paraformaldehyde in PBS, or other conventional fixatives, may be used.

Thereafter, the cell populations are exposed to a substrate for β-galactosidase activity. In one embodiment, this solution is a dye or substrate that produces a color phase contrast in response to increasing or decreasing amounts of β-galactosidase. In one embodiment, the substrate is 5-bromo-4-chloro-3-indolyl-β-D-galactopyranoside (X-Gal). In another embodiment, the substrate is di-β-D-galactopyranoside (C12FDG). The substrates may be employed in any suitable solution. Suitable solutions can contain sodium phosphate (Na₂HPO₄), K₃Fe(CN)₆, and K4Fe(CN)₆, among other additives common for such solutions. Still other substrates may be employed for this purpose; this method is not limited by the selection of the β-galactosidase dyes or substrate. The cell populations are further exposed to a fluorescent nuclear stain. In this method the nuclei serve as a surrogate for cell number. Conventional nuclear stains may be employed for this purpose. In one embodiment, the nuclear stain is DAPI. In another embodiment, a suitable nuclear stain is a Hoechst stain. Still other known nuclear stains, including, without limitation, Hematoxylin, methylene blue, neutral/toluoylene red, Nile blue, or Safranin, may be similarly employed in this step.

Once the cell populations are properly prepared and exposed to the reagents of the method, the cells are analyzed, e.g., in the multi-well format, with a high-content microscopic imaging system that detects fluorescence of stained nuclei and color phase contrast produced by the amount of β-galactosidase in each well. The imaging system acquires multiple fluorescence and phase contrast images from each well, applies the control threshold, and averages the values. In one embodiment, the number of multiple images is 4. However, other multiples may be useful. As described in the examples below, a useful imaging system is the IMAGEXPRESS MICRO high-content imaging system (Molecular Devices LLC, Sunnyvale, Calif.), or a similar instrument.

The data generated by this imaging system is analyzed by use of an algorithm, such as provided below, to generate a threshold based on untreated negative or positive control cells which determines significant changes in the number of stained nuclei and amount of β-galactosidase. Test compounds that inhibited senescence were identified by the following algorithm, using the abbreviations TC (test compound), x (x=any given designation of test compound, e.g. TC_{AG1478}), Nctrl (average number of cells of control), NTCx (average number of cells exposed to TC), BGPPctrl (SA-β-gal positive area in the control wells), BGPPTCx (SA-β-gal area for TC):
TC prevented senescence if NTCx>[Nctrl+Standard Deviation of Nctrl] and at the same time BGPPTCx<[BGPPctrl−Standard Deviation of BGPPctrl].
   The average number of nuclei and SA-β-gal area from each test compound is normalized to DMSO, and these values are averaged between replicates.

An optional plot of the normalized average number of stained nuclei vs the normalized amount of β-galactosidase area in each well can be created from the results of the algorithm employing the threshold to create a visual representation of the algorithm. Thereafter, from an analysis of the scatterplot or algorithm, the compounds in the wells that produce the desired conditions of increased or decreased cell senescence are identified.

In another aspect, an additional method step includes validating these results by further exposing cells treated with the identified compounds producing the desired conditions with a fluorescent nuclear dye for visualization of senescence-associated heterochromatin foci (SAHF). In one embodiment, this step involves measuring the percentage of SAHF following treatment, which increases the reliability of the method for identifying senescent cells.

Identification of the cells exposed to test compounds that modulate senescence is thereafter accomplished by selecting those cells on the scatterplot (or identified via the results of the algorithm) showing a decrease in expression of the senescence marker with an increase in cell number as compared to the negative control or threshold. Cells meeting these conditions have been treated with a test compound that promotes cell growth and suppresses cell senescence. Such a test compound, when used on normal healthy cells in this method, is useful in delaying the aging process of normal healthy cells. Similarly, such a test compound when used on normal healthy cells in this method may be useful as a tumor suppressor.

In another embodiment, identification of the cells exposed to test compounds that modulate senescence is thereafter accomplished by selecting those cells on the scatterplot (or via the algorithm) showing an increase in expression of the senescence marker and a decrease in cell number as compared to the negative control. Cells meeting these conditions have been treated with a test compound that inhibits cell growth and promotes senescence. Such a test compound when used cancer cells in this method may be useful in the treatment of cancer.

As described below, an illustrative embodiment of this method was performed by employing this high-content microscopic imaging system based screen in primary human fibroblasts undergoing senescence induced by oncogenic RAS (H-RasG12V). A library of 160 well-characterized, small molecule kinase inhibitors was screened to determine compounds that inhibited or suppressed OIS, e.g., in the context of oncogenic RAS overexpression. In one embodiment, the compounds were screened for suppressors of senescence induced by oncogenic RAS. Importantly, the identified compounds can elucidate other proteins involved in mediating OIS and novel tumor suppressors. Utilizing this newly developed platform, the inventors identified from a kinase inhibitor library, 17 kinase inhibitors as suppressors of oncogenic RAS-induced senescence. The majority of these compounds target multiple kinases¹⁸. Identified compounds were subsequently validated and confirmed using a third marker of senescence, senescence-associated heterochromatin foci. All 17 inhibitors were individually validated and 16 out of 17 these were confirmed using SAHF staining as an additional marker of senescence. In addition, one of the inhibitors identified (AMPK Compound C, CAS #855405-64-3) has been reported to prevent senescence¹⁶, further demonstrating the robustness of the newly established assay.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1

### Materials and Methods

A. Tissue Culture.

Primary diploid fibroblasts (IMR90) were cultured according to the American Type Culture Collection (ATCC). Experiments were performed with IMR90 that were between 25 and 36 population doublings (PD).

B. Plasmids and Retrovirus.

pBABE-H-RasG12V was obtained from Addgene. Retrovirus production and transduction has previously been described 10. Phoenix cells were used to facilitate viral packaging (Dr. Gary Nolan, Stanford University).

C. Screen Setup.

We use a model system for DIS that involves overexpressing an activated Ras in normal human fibroblast (IMR90). Upon expression of Ras, these cells undergo senescence as measured by increased β-galactosidase activity. IMR90 cells are infected with retrovirus control (496) or Ras (493). IMR90 cells were transduced over two days (Day −1 and 0) with pBABE-HRasG12V. Cells were then plated into a 96-well plate at a predetermined density (1,000 cells/well) and selected with puromycin (1 ug/mL) for two days (Day 1 and 2). The initial number of cells per well (1,000) was optimized to avoid confluence-induced growth inhibition. Allowing one day for cells to attach to the plate, at the end of day 2, cells were treated with kinase inhibitors (KI) by pin transfer at ˜250 nM. Allowing the cells to undergo senescence (10 days after puromycin selection), on day 9, cells were subjected to a quantitative SA-β-gal assay (described below) and stained with DAPI to visualize nuclei, which allowed for quantification of cell number.

D. Senescence Assay.

Senescence-associated β-galactosidase (SA-β-Gal) assay has previously been described⁹. Briefly, cells were fixed in 2% formaldehyde and 0.2% glutaraldehyde and washed with phosphate-buffered saline. Staining solution [40 mM Na₂HPO₄, 150 mM NaCl, 2 mM MgCl₂, 5 mM K₃Fe(CN)₆, 5 mM K₄Fe(CN)₆, 1 mg/mL X-gal] was added with a multichannel pipette, and cells were incubated for 24 hrs. Cells were stained with DAPI (0.15 μg/mL) to visualize nuclei.

For the screen, four fluorescence and four phase contrast images were acquired from each well of the 96-well plates with an ImageXpress Micro high-content imaging system (Molecular Devices, Sunnyvale Calif.) using the 10× objective. Note that at 10× magnification SAHF are indistinguishable. Phase contrast images were used to record SA-β-gal staining. Fluorescence images were used to record DAPI staining of cell nuclei. Images were analyzed utilizing the MultiWavelength Scoring module in METAXPRESS image analysis software (Molecular Devices).

Briefly, based on preliminary experiments with control and RAS-expressing cells, a threshold was determined at which to define positive beta-gal pixels. This threshold was applied to all of the phase contrast images (FIG. 2A, orange pixels), and the masked area for each image was systematically quantified. DAPI positive nuclei were counted with METAXPRESS image analysis software by restricting counts to circular objects with a diameter greater than 1 micron.

ACUITYXPRESS software was used for informatics processing and data visualization. The four images were quantified and the values for each SA-β-gal area and nuclei number were averaged.

We determined the average number of cells (Nctrl) and SA-β-gal area (BGPPctrl) for IMR90s transduced with pBABE-H-RasG12V treated with DMSO vehicle control, which served as a positive base value for comparison. The average number of nuclei and SA-β-gal area from each of the 160 kinase inhibitors was normalized to DMSO, and these values were averaged between replicates. Kinase inhibitors (KI) that inhibited senescence were identified by the following equation: KI prevented senescence if NKIx>[Nctrl+Standard Deviation of Nctrl] and at the same time BGPPKIx<[BGPPctrl−Standard Deviation of BGPPctrl].

All 17 active compounds identified as hits in the screen, along with an inactive control compound (CAS #648449-76-7), were individually examined for SA-β-gal activity and SAHF formation. Cells with β-galactosidase (blue) positivity within the cytosol were considered positive. Cells were stained with DAPI to visualize nuclei and SAHF. Cells with punctate spots within the nuclear compartment were considered SAHF positive. Cells were imaged using a Nikon Eclipse Ni fluorescence microscope as previously described¹⁵. Three different fields of cells were counted (total cells counted >300). These experiments were performed in triplicate. Data from validation experiments is shown as a percentage of SA-β-gal or SAHF positive cells.

E. Statistical Analysis.

Statistical significance was determined by t-test using GraphPad statistical software (La Jolla, Calif.). Values were considered significant if p<0.05.

F. Kinase Inhibitor Library.

The INHIBITORSELECT kinase inhibitor library, which contained 160 well-characterized kinase inhibitors, was purchased from EMD Millipore Compounds were maintained in DMSO. Inhibitors were added to the growth medium of cells growing in 96-well microplates by pin transfer. Eight wells of cells in each plate treated with DMSO (vehicle control) were included as negative controls. To avoid plate location-specific effects, these cells were spread across the plate.

### Example 2

### Development of a High-Throughput Platform for Identification of Small Molecule Modulators of Senescence Induced by Oncogenic RAS

Ectopic expression of activated oncogenes such as RAS is a standard approach to induce cellular senescence in a synchronized manner in primary mammalian cells³. Thus, we ectopically expressed oncogenic H-RASG12V by retroviral transduction in primary human fibroblasts (IMR90 cells) to induce senescence. Senescence phenotypes, such as SA-β-gal activity, typically take several days to develop¹⁴. SA-β-gal activity in the cytoplasm is considered a universal marker of cellular senescence⁹ and can be visualized by phase contrast microscopy. In addition, a definitive marker of senescence is a decreased proliferation¹⁶.

We sought to develop a high-throughput platform for screening small molecule modulators of senescence. In an initial pilot experiment, using METAXPRESS image analysis, a threshold of SA-β-gal intensity was established to define a SA-β-gal positive pixel. This threshold was subsequently used in the large-scale screen to apply a “mask” to the four phase contrast images, and the positive pixel area per image was determined (FIG. 2A, orange area). Importantly, in control wells, we observed a significant and robust difference in SA-β-gal area between non-RAS expressing control and RAS-expressing cells (FIG. 2B). We also observed a significant reduction in the number of nuclei in RAS-expressing cells compared to control cells (FIGS. 2C and 2D). The average SA-β-gal area and number of nuclei for the positive control (DMSO vehicle treated RAS-expressing cells) were used as a baseline to compare with the kinase inhibitor treated cells.

It is well established that a cascade of kinases plays a role in regulating oncogenic RAS induced senescence¹⁷. We therefore validated our assay using a library of 160 well-characterized kinase inhibitors screened in a 96-well plate format¹⁸. The screen was performed by treating RAS-transduced cells with 250 nM of each of the 160 kinase inhibitors or vehicle control (DMSO) (FIG. 1). This concentration was chosen based on typical concentrations at which these compounds are utilized in cell-based assays. Furthermore, it is known that cell density affects SA-β-gal activity. To limit the potential non-specific effects of cell density on SA-β-gal expression, a range of cell concentrations were tested during assay development to avoid cell confluence over the course of the assay.

Typically, 1,000 RAS-overexpressing cells were inoculated per well. We utilized a time-point of 9 days following RAS transduction to assess OIS based on our previous work¹⁴. Eight wells of vehicle control were included in each plate for comparison. Non-RAS-infected cells were included as an additional control. Cells in the 96-well plates were then fixed, and markers of senescence were examined. Accordingly, we stained the fixed cells for SA-β-gal activity and with a fluorescent DNA dye (DAPI) to visualize nuclei, which was used as a surrogate for cell number. Four phase contrast and fluorescence images were taken of each well and systematically quantified and averaged. This assay allows us to measure the expression of a senescence marker simultaneously with cell number to reflect changes in proliferation in the same population of cells. To limit the possibility that the decrease in expression of SA-β-gal activity was not due to a decrease in cell proliferation, we focused on kinase inhibitors that significantly suppressed SA-β-gal activity but did not decrease cell proliferation.

Averages of SA-β-gal area and nuclei number from replicate experiments were compiled and normalized to the mean of vehicle (DMSO) control wells. These data were graphed on a scatter plot (FIG. 3). Hits were identified as compounds that reduced SA-β-gal area by at least one standard deviation below the mean of DMSO control and conversely increased cell number (nuclei) above one standard deviation of the DMSO control mean (FIG. 3, bottom right quadrant, gray or blue dots). According to these criteria, 17 compounds significantly suppressed SA-β-gal area while maintaining nuclei number in RAS-expressing cells (compounds are listed in Table 1).

Table 1 lists the kinase inhibitors that suppressed Ras-induced senescence. Compound identification number is the Chemical Abstract Service (CAS) number.

TABLE 1

| CAS# | Compound Name |
|---|---|
| 722617-28-8 | AGL2043 |
| 612847-09-3 | Akt Inhibitor VIII |
| 866405-64-3 | AMPK Compound C |
| 879127-16-9 | Aurora Kinase Inhibitor III |
| 516480-79-8 | Chk2 Inhibitor II |
| 120166-69-0 | Diacylglycerol Kinase Inhibitor II |
| 327036-89-5 | GSK3β Inhibitor I |
| 404828-08-6 | GSK-3 Inhibitor XIII |
| 139298-40-1 | KN-93 |
| 658084-23-2 | Met Kinase inhibitor |
| 219138-24-6 | P38 MAPK Inhibitor |
| 347155-76-4 | PDGFR Tyrosine Kinase Inhibitor V |
| 371935-74-9 | PI-103 |
| 257879-35-9 | PKC β Inhibitor |
| 172747-50-1 | SB 202474 |
| 175178-82-2 | AG 1478 |
| 269390-69-4 | VEGFR Tyrosine Kinase Inhibitor II |

### Example 3

### Validation of Identified Compounds as Suppressors of Oncogene-Induced Senescence

Recapitulating the experimental design of the screen, we individually treated RAS-expressing cells with DMSO vehicle control or with 250 nM of the 17 identified inhibitors. As an additional control, RAS-expressing cells were also treated with a kinase inhibitor (CAS #648449-76-7) that had no effect on SA-β-gal area or cell number based on initial screening (FIG. 3, arrow). These cells were then utilized in the SA-β-gal assay to examine changes in expression of SA-β-gal activity. Upon manual quantification of SA-β-gal positive cells, we discovered that 16 of 17 inhibitors significantly reduced the expression of SA-β-gal activity in our validation experiments (FIGS. 4A-4B and Table 2), representing a false positive rate of 1/17 (5.9%).

To further confirm senescence suppression by these compounds, we next assayed their effect using an independent marker of senescence, senescence-associated heterochromatin foci (SAHF), which are domains of punctate DAPI stain in the nuclei of senescent cells (FIG. 5A, white arrowheads)¹². Toward this goal, RAS-expressing cells were treated with the 17 kinase inhibitors identified in the initial screen, a negative control inhibitor (CAS #648449-76-7) or DMSO. After 9 days of culture, we stained the cells with DAPI to visualize the formation of SAHF. In this independent validation, we discovered that 16 of 17 inhibitors significantly inhibited oncogenic RAS-induced SAHF formation. Interestingly, the inhibitor that failed to prevent SAHF formation (PI-103; CAS #371935-74-9) was not the same as the inhibitor that failed to reduce SA-β-gal activity (Akt Inhibitor VIII; CAS #612847-09-3) (FIG. 5 A-B and Table 2). The discrepancy was not unexpected as there is evidence to suggest that SAHF formation is not always necessary for cells to undergo senescence¹⁹. Thus, this compound confirms that SAHF formation can be uncoupled from SA-β-gal activity and may represent a novel tool to understand this phenomenon. In summary, we validated 16 out of the 17 compounds identified from the initial screening as bona fide suppressors of oncogenic RAS-induced senescence, demonstrating that this assay is a robust platform for identifying regulators of OIS.

Table 2 shows the validation of 17 identified senescence-suppressing compounds. Other than the control, all of the tested compounds are +H-Ras^{G12V}. Percentage of SA-β-gal and SAHF positive cells is shown with indicated standard deviation (SD). Total cells with SA-β-gal positive cells or SAHF positive cells were counted in three separate fields (totals cells counted >300). Experiments were performed in triplicate. p-value determined by t-test.

TABLE 2

| Compound | % SA-β-Gal + Cells | | | % SAHF + Cells | | |
|---|---|---|---|---|---|---|
| Tested | Avg | SD | p-value | Avg | SD | p-value |
| Control | 2.5 | 0.0 | N/A | 1.4 | 0.4 | N/A |
| DMSO | 20.0 | 4.6 | N/A | 36.4 | 5.7 | N/A |
| 648449-76-7 | 20.7 | 5.0 | 0.8340 | 26.9 | 6.8 | 0.1381 |
| 175178-82-2 | 3.8 | 0.8 | 0.0038 | 11.5 | 2.1 | 0.0020 |
| 269390-69-4 | 4.1 | 1.6 | 0.0047 | 13.0 | 1.2 | 0.0022 |
| 866405-64-3 | 4.4 | 2.5 | 0.0065 | 15.9 | 4.3 | 0.0077 |
| 371935-74-9 | 4.7 | 1.6 | 0.0055 | 43.0 | 9.2 | 0.3487 |
| 658084-23-2 | 5.0 | 1.7 | 0.0060 | 14.7 | 6.8 | 0.0133 |
| 120166-69-0 | 5.2 | 3.1 | 0.0097 | 10.4 | 1.8 | 0.0016 |
| 257879-35-9 | 5.5 | 2.7 | 0.0091 | 8.3 | 3.5 | 0.0089 |
| 879127-16-9 | 5.7 | 2.7 | 0.0096 | 15.8 | 3.5 | 0.0060 |
| 219138-24-6 | 5.8 | 1.2 | 0.0064 | 16.3 | 1.7 | 0.0042 |
| 327036-89-5 | 6.5 | 1.4 | 0.0080 | 7.8 | 4.6 | 0.0025 |
| 172747-50-1 | 6.7 | 0.8 | 0.0077 | 10.4 | 3.8 | 0.0027 |
| 347155-76-4 | 6.7 | 2.8 | 0.0126 | 16.3 | 4.7 | 0.0092 |
| 516480-79-8 | 7.4 | 1.2 | 0.0099 | 13.2 | 4.2 | 0.0047 |
| 139298-40-1 | 8.1 | 1.1 | 0.0120 | 12.3 | 1.8 | 0.0115 |
| 404828-08-6 | 8.2 | 1.6 | 0.0138 | 7.0 | 1.9 | 0.0011 |
| 22617-28-8 | 10.9 | 2.8 | 0.0422 | 10.2 | 6.1 | 0.0056 |
| 612847-09-3 | 21.3 | 1.0 | 0.7326 | 11.3 | 0.4 | 0.0016 |

This data establishes a high-throughput screening platform for suppressors of senescence and identify several compounds as suppressors of oncogene-induced senescence.

A previously described a screen for modulators of stress-induced senescence in prostate cancer cells²² utilized SA-β-gal as a senescence output but were only able to perform manual quantification of SA-β-gal cells in conditions that significantly altered cell number. In contrast, the method described herein utilizes a high-content imaging system to quantify both the number of nuclei and measure SA-β-gal activity. This newly developed assay allows us to examine SA-β-gal activity in a high-throughput manner.

The proof-of-principle kinase inhibitor screen described herein identified compounds that suppressed senescence, which was determined by a decrease in SA-β-gal activity. In validation studies, we observed a discrepancy between kinase inhibitors that prevented SA-β-gal versus those that inhibited SAHF. In addition, the high throughput approach utilized in this assay allows for the examination enhancers of OIS.

While senescence is known to be an important tumor suppression mechanism during tumor initiation and progression, it may also be an important mechanism for developing cancer therapeutics. For example, reactivation of the tumor suppressor p53 in murine liver and sarcoma models triggers cellular senescence and the associated tumor regression due to activation of the innate immune response²³. Likewise, restoration of noncanonical Wnt signaling in ovarian cancer cells suppresses tumorigenesis by inducing cellular senescence²⁴. Thus, small molecule inhibitors that promote senescence of cancer cells are likely cancer therapeutics.

In conclusion, the inventors established a high-throughput screening platform for identifying small molecule regulators of cellular senescence and identified compounds that are suppressors of senescence induced by oncogenic RAS. In addition, their validation reveals the feasibility of using this newly developed screen to identify modulators of senescence, including oncogene-induced senescence.

Technical and scientific terms used herein have the same meaning as commonly understood by one of ordinary skill in the art to which this invention belongs and by reference to published texts, which provide one skilled in the art with a general guide to many of the terms used in the present application. Any definitions are provided for clarity only and are not intended to limit the claimed invention.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “a test compound,” is understood to represent one or more test compounds. As such, the terms “a” (or “an”), “one or more,” and “at least one” is used interchangeably herein.

Each and every patent, patent application, including U.S. Provisional patent application No. 61/789,328, and publication, including publications listed below, and publically available peptide sequences cited throughout the disclosure, is expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

### REFERENCES

1. Chen, Z. et al, Crucial role of p53-dependent cellular senescence in suppression of Pten-deficient tumorigenesis. Nature 2005, 436(7051), 725-30;
2. Lin, A. W. et al, Premature senescence involving p53 and p16 is activated in response to constitutive MEK/MAPK mitogenic signaling. Genes Dev 1998, 12 (19), 3008-19;
3. Serrano, M. et al, Oncogenic ras provokes premature cell senescence associated with accumulation of p53 and p161NK4a. Cell 1997, 88 (5), 593-602.
4. Kuilman, T. et al, The essence of senescence. Genes Dev 2010, 24 (22), 2463-79.
5. Goal, V. K. et al, Melanocytic nevus-like hyperplasia and melanoma in transgenic BRAFV600E mice. Oncogene 2009, 28 (23), 2289-98;
6. Bauer, J. et al. Congenital melanocytic nevi frequently harbor NRAS mutations but no BRAF mutations. J Invest Dermatol 2007, 127 (1), 179-82;
7. Denoyelle, C. et al. Anti-oncogenic role of the endoplasmic reticulum differentially activated by mutations in the MAPK pathway. Nat Cell Biol 2006, 8 (10), 1053-63.
8. Schubbert, S. et al., Hyperactive Ras in developmental disorders and cancer. Nat Rev Cancer 2007, 7 (4), 295-308.
9. Dimri, G. P. et al., A biomarker that identifies senescent human cells in culture and in aging skin in vivo. Proc Natl Acad Sci USA 1995, 92 (20), 9363-7.
10. Itahana, K. et al, Methods to detect biomarkers of cellular senescence: the senescence-associated beta-galactosidase assay. Methods Mol Biol 2007, 371, 21-31.
11. Caldwell, M. E. et al. Cellular features of senescence during the evolution of human and murine ductal pancreatic cancer. Oncogene 2012, 31 (12), 1599-608.
12. Narita, M. et al. Rb-mediated heterochromatin formation and silencing of E2F target genes during cellular senescence. Cell 2003, 113 (6), 703-16.
13. Zhang, R. et al. Molecular dissection of formation of senescence-associated heterochromatin foci. Mol Cell Biol 2007, 27 (6), 2343-58.
14. Tu, Z. et al. Oncogenic RAS regulates BRIP1 expression to induce dissociation of BRCA1 from chromatin, inhibit DNA repair, and promote senescence. Dev Cell 2011, 21 (6), 1077-91.
15. Zhang, R. et al. Formation of MacroH2A-containing senescence-associated heterochromatin foci and senescence driven by ASF1a and HIRA. Dev Cell 2005, 8 (1), 19-30.
16. Hayflick, L., The cell biology of aging. Clin Geriatr Med 1985, 1 (1), 15-27.
17. Sun, P. et al. PRAK is essential for ras-induced senescence and tumor suppression. Cell 2007, 128 (2), 295-308.
18. Anastassiadis, T. et al. Comprehensive assay of kinase catalytic activity reveals features of kinase inhibitor selectivity. Nat Biotechnol 2011, 29 (11), 1039-45.
19. Kosar, M. et al. Senescence-associated heterochromatin foci are dispensable for cellular senescence, occur in a cell type- and insult-dependent manner and follow expression of p16(ink4a). Cell Cycle 2011, 10 (3), 457-68;
20. Di Micco, R. et al. Interplay between oncogene-induced DNA damage response and heterochromatin in senescence and cancer. Nat Cell Biol 2011, 13 (3), 292-302.
21. Zu, Y. et al. SIRT1 promotes proliferation and prevents senescence through targeting LKB1 in primary porcine aortic endothelial cells. Circ Res 2010, 106 (8), 1384-93.
22. Ewald, J. A. et al. A high throughput method to identify novel senescence-inducing compounds. J Biomol Screen 2009, 14(7), 853-8.
23. Xue, W. et al. Senescence and tumour clearance is triggered by p53 restoration in murine liver carcinomas. Nature 2007, 445 (7128), 656-60.
24. Bitter, B. G. et al., Wnt5a suppresses epithelial ovarian cancer by promoting cellular senescence. Cancer Res 2011, 71 (19), 6184-94.
25. Dhomen, N. et al. Oncogenic Braf induces melanocyte senescence and melanoma in mice. Cancer Cell 2009, 15 (4), 294-303;
26. Michaloglou, C. et al. BRAF E600-associated senescence-like cell cycle arrest of human naevi. Nature 2005, 436 (7051), 720-4.

1. A high-throughput method for identifying a compound that modulates cell senescence comprising simultaneously measuring in a cell population exposed to a test compound, the expression of a senescence marker and cell number; and determining from said simultaneous measurements whether the test compound increases or decreases cell senescence.

2. The method according to claim 1, wherein the cell population is contained in a multi-well format and wherein each well contains a single test compound.

3. The method according to claim 1, further comprising treating or incubating the cells in each well sequentially, with
i. a single test compound per well for a time suitable for modulation of senescence in the wells;
ii. a dye that produces a color phase contrast in response to increasing amounts of β-galactosidase, and
iii. a fluorescent nuclear stain.

4. The method according to claim 1, further comprising:
analyzing the cells in the multi-well format with a high-content microscopic imaging system that detects fluorescence of stained nuclei and color phase contrast produced by the amount of β-galactosidase in each well;
generating through use of an algorithm a threshold based on untreated negative or positive control cells for determining significant changes in the number of stained nuclei and amount of β-galactosidase;
identifying from the algorithm the compounds in the wells that produce the desired conditions of increased or decreased cell senescence.

5. The method according to claim 1, further comprising further exposing cells treated with the identified compounds producing the desired conditions with a fluorescent nuclear dye for visualization of senescence-associated heterochromatin foci (SAHF).

6. The method according to claim 4, further comprising:
generating a scatterplot of the average number of stained nuclei vs the amount of β-galactosidase produced in each well using said threshold as a visual representation of the algorithm results, wherein the compounds are identified from the scatterplot.

7. The method according to claim 1, which is automated or performed by a computer.

8. The method according to claim 1, wherein the cells are primary mammalian cells transduced with an oncogene, mammalian cancer cells that naturally express an oncogene, or naturally senescent healthy mammalian cells.

9. The method according to claim 8, wherein the mammalian cells are fibroblasts, hepatocytes or epithelial cells.

10. The method according to claim 8, wherein the cancer cell is a melanoma cell.

11. The method according to claim 8, wherein the oncogene is selected from RAS, B-raf, PI3K/AKT or loss of PTEN.

12. The method according to claim 3, wherein the cells containing the single test compound are fixed and washed prior to treatment or incubation with the dye (ii).

13. The method according to claim 3, wherein the dye (ii) is a solution comprising a substrate for β-galactosidase activity.

14. The method according to claim 13, wherein the substrate is 5-bromo-4-chloro-3-indolyl-β-D-galactopytranoside (X-Gal) or di-β-D-galactopyranoside (C12FDG).

15. The method according to claim 3, wherein the nuclear stain is DAPI, a Hoechst stain, Hematoxylin, methylene blue, neutral/toluoylene red, Nile blue, or Safranin.

16. The method according to claim 3, wherein the imaging system acquires multiple fluorescence and phase contrast images from each well, applies the control threshold, and averages the values.

17. The method according to claim 16, wherein the multiple images are 4.

18. The method according to claim 1, wherein the test compounds are small molecules.

19. The method according to claim 1, comprising:
(a) identifying the test compound as one that promotes cell growth and suppresses cell senescence when the cells exposed to the test compound show a decrease in expression of the senescence marker and an increase in cell number as compared to the negative control; or
(b) identifying the test compound as one that inhibits cell growth and promotes cell senescence when cells exposed to the test compound show an increase in expression of the senescence marker and a decrease in cell number as compared to the negative control.

20. The method according to claim 19, comprising using the test compound of (a) in a treatment for delaying the aging process of normal healthy cells or using the test compound of (a) as a tumor suppressor when the cells exposed to the compound are healthy cells; or using the test compound of (b) in the treatment of cancer when the cells exposed to the test compound are cancer cells.
